(19) United States
(12) Patent Application Publication
(10) Pub. No.: US 2011/0040227 A1
(43) Pub. Date: Feb. 17, 2011

(54) Wrap Apparatus and Method of Wrapping a Body Part with a Wrap Apparatus

(76) Inventor: Steven Magri, Hudson, NH (US)

Correspondence Address: HAYES, SOLOWAY P.C., 175 CANAL STREET, MANCHESTER, NH, 03101, US

(21) Appl. No.: 12/852,891

(22) Filed: Aug. 9, 2010

Related U.S. Application Data
(60) Provisional application No. 61/232,829, filed on Aug. 11, 2009.

(51) Int. Cl. A61F 13/10

(52) U.S. Cl. 602/62

(57) ABSTRACT

A wrap apparatus and method of wrapping a body part with a wrap apparatus includes an elongated elastic fabric material having a first end, a second end and a middle portion located between the first end and second end. A loop is integral with the first end of the elongated elastic fabric material. A retention device is located proximate to the second end of the elongated elastic fabric material, wherein the retention device is removably fixable to the middle portion of the elongated elastic fabric material.

### CROSS REFERENCE TO RELATED APPLICATION

This application claims benefit of U.S. Provisional Application Ser. No. 61/232,829 filed Aug. 11, 2009, the entire disclosure of which is incorporated herein by reference.

### FIELD

The present disclosure is generally related to a wrap apparatus, and more particularly is related to a system and method for wrapping a body part with a wrap apparatus.

### BACKGROUND

ACE™ brand bandages, and their like, have been around since approximately 1914. The most common of the bandages consists of an elastic band of material that is wrapped around a body part multiple times and cinched with a clasp. Wrapping the elastic band tightly around a body part may combat swelling of a muscle, or the like, by providing distributed pressure about the muscle. The elastic band may also be wrapped around an ice pack or heated pack, which is positioned against an injured body part, where the elastic band secures the ice/heated pack against the injured body part.

Application of the elastic band includes inherent difficulties. For instance, it may be difficult and/or awkward for an individual to wrap the band about their own body part, particularly if they are trying to wrap the band around one of their hands, arms, or shoulders, as the elastic band is nearly impossible to wrap using a single hand. The difficulty is increased if trying to include the ice/heated pack, as it is difficult to hold the pack in place while using two hands to wrap the elastic band, particularly if the pack is placed against a hard to reach place, such as the lower back or a shoulder blade. Failure to apply the elastic band with adequate tightness about the pack will cause the pack to slip out, particularly if the wearer chooses not to remain immobile after wrapping the elastic band.

Thus, a heretofore unaddressed need exists in the industry to address the aforementioned deficiencies and inadequacies.

### SUMMARY

Embodiments of the present disclosure provide a system and method of wrapping a body part with a wrap apparatus. Briefly described, in architecture, one embodiment of the system, among others, can be implemented as follows. The system contains an elongated elastic fabric material having a first end, a second end and a middle portion located between the first end and second end. A loop is integral with the first end of the elongated elastic fabric material. A retention device is located proximate to the second end of the elongated elastic fabric material, wherein the retention device is removably fixable to the middle portion of the elongated elastic fabric material.

The present disclosure can also be viewed as providing methods for wrapping a body part with a wrap apparatus. In this regard, one embodiment of such a method, among others, can be broadly summarized by the following steps: sliding the body part through a loop positioned integral with the first end of an elongated elastic fabric material; wrapping a middle portion of the elongated elastic fabric material about the body part; and removably fixing a second end of the elongated elastic fabric material proximate to the middle portion of the elongated elastic fabric material with a retention device located proximate to the second end of the elongated elastic fabric material.

The present disclosure can also be viewed as providing methods for wrapping a body part with a wrap apparatus. In this regard, one embodiment of such a method, among others, can be broadly summarized by the following steps: positioning a middle portion of an elongated elastic fabric material proximate a body part; sliding a second end of the elongated elastic fabric material through a loop positioned integral with the first end of an elongated elastic fabric material; pulling the second end of the elongated elastic fabric material, thereby compressing the middle portion of the elongated elastic fabric material around the body part; and removably fixing the second end of the elongated elastic fabric material proximate to the middle portion of the elongated elastic fabric material with a retention device located proximate to the second end of the elongated elastic fabric material.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale. Instead emphasis is being placed upon illustrating clearly the principles of the present disclosure. Moreover, in the drawings, like reference numerals designate corresponding parts throughout the several views.

FIG. 1 is an illustration of a prospective view of a wrap apparatus, in accordance with a first exemplary embodiment of the present disclosure.

FIG. 2A is an illustration of a prospective view of a wrap apparatus on a body part in a first exemplary way, in accordance with the first exemplary embodiment of the present disclosure.

FIG. 2B is an illustration of a prospective view of a wrap apparatus on a body part in a first exemplary way, in accordance with the first exemplary embodiment of the present disclosure.

FIG. 2C is an illustration of a prospective view of a wrap apparatus on a body part in a first exemplary way, in accordance with the first exemplary embodiment of the present disclosure.

FIG. 3A is an illustration of a prospective view of a wrap apparatus on a body part in a second exemplary way, in accordance with the first exemplary embodiment of the present disclosure.

FIG. 3B is an illustration of a prospective view of a wrap apparatus on a body part in a second exemplary way, in accordance with the first exemplary embodiment of the present disclosure.

FIG. 3C is an illustration of a prospective view of a wrap apparatus on a body part in a second exemplary way, in accordance with the first exemplary embodiment of the present disclosure.

FIG. 3D is an illustration of a prospective view of a wrap apparatus on a body part in a second exemplary way, in accordance with the first exemplary embodiment of the present disclosure.

FIG. 4 is an illustration of a prospective view of a wrap apparatus having a thermal pack, in accordance with a second exemplary embodiment of the present disclosure.

FIG. 5 is an illustration of a top view of a wrap apparatus having a thermal pack, in accordance with the second exemplary embodiment of the present disclosure.

FIG. 6 is an illustration of a prospective view of a wrap apparatus, in accordance with a third exemplary embodiment of the present disclosure.

FIG. 7 is an illustration of a flowchart illustrating a method of wrapping a body part with a wrap apparatus of FIG. 1, in accordance with the first exemplary embodiment of the present disclosure.

FIG. 8 is an illustration of a flowchart illustrating a method of wrapping a body part with a wrap apparatus of FIG. 1, in accordance with the first exemplary embodiment of the present disclosure.

### DETAILED DESCRIPTION

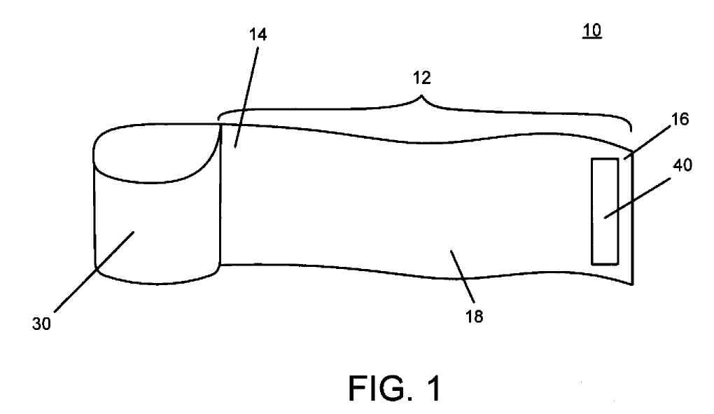

FIG. 1 is an illustration of a prospective view of a wrap apparatus 10, in accordance with a first exemplary embodiment of the present disclosure. The wrap apparatus 10 includes an elongated elastic fabric material 12 having a first end 14, a second end 16 and a middle portion 18 located between the first end 14 and second end 16. A loop 30 is integral with the first end 14 of the elongated elastic fabric material 12. A retention device 40 is located proximate to the second end 16 of the elongated elastic fabric material 12, wherein the retention device 40 is removably fixable to the middle portion 18 of the elongated elastic fabric material 12.

The wrap apparatus 10 may commonly be used to provide compression to a body part in a medical or therapeutic situation, such as with a sprained wrist, although the wrap apparatus 10 may have a variety of other uses as well. The wrap apparatus 10 is constructed from at least one elongated elastic fabric material 12. The elongated elastic fabric material 12 may be composed of a fabric that has an elastic property to it, such as a fabric that is embedded with an elastic element to provide elasticity within the wrap apparatus 10. This may include any type of base materials, such as a cotton or polyester fabric with embedded elastic elements. Common examples may be seen with Ace™ brand bandages from the 3M Corporation or ‘Loop Stretch’ from East Tech Corporation. The elongated elastic fabric material 12 may be placed proximate to the skin of a user when the wrap apparatus 10 is in use, so it is preferable for the elongated elastic fabric material 12 to be latex-free to minimize skin irritations. The elongated elastic fabric material 12 may also be free of other possible irritants, which may be specified depending on a particular use of the wrap apparatus 10. The wrap apparatus 10 may have an affixed identification tag to identify a characteristic of the wrap apparatus 10, such a composition, a place of manufacture, a company of manufacture, or any other characteristic of the wrap apparatus 10.

As is shown in FIG. 1, the elongated elastic fabric material 12 has a length measurement that is considerably greater than a width measurement. The wrap apparatus 10 is designed and envisioned to wrap around a human body part, such as an arm, wrist, leg, torso or any other body part. The elongated elastic fabric material 12 may preferably have a width measurement of between 3 and 4 inches, but may also include any other width measurement. The length of the elongated elastic fabric material 12 may be varied depending on the intended use of the wrap apparatus 10. For example, the elongated elastic fabric material 12 may commonly be 2 feet to 4 feet long, although any length of the elongated elastic fabric material 12 is considered within the scope of this disclosure. It is noted that the wrap apparatus 10 may be wrapped proximate to any body part, and may be sized accordingly to provide appropriate coverage. For example, a wrap apparatus 10 used with a user's wrist or ankle may be sized smaller than a wrap apparatus 10 used with a user's torso or back.

As shown, the elongated elastic fabric material 12 has a first end 14, a second end 16 and a middle portion 18 located between the first end 14 and second end 16. The first end 14 is generally characterized as the end of the elongated elastic fabric material 12 that is proximate to the loop 30. The second end 16 is generally characterized as the end of the elongated elastic fabric material 12 that is distal from the loop 30. The loop 30 may be formed with the same material as the elongated elastic fabric material 12, such as by sewing the elongated elastic fabric material 12 over itself. Alternatively, the loop 30 may be constructed from a different material and integrally affixed to the first end 14 of the elongated elastic fabric material 12.

The loop 30 may be permanently affixed to the elongated elastic fabric material 12 from the elongated elastic fabric material 12 depending on design. A variety of different sized loops 30 or one universally sized loop 30 may also be used with the wrap apparatus 10 depending on the intended use of the wrap apparatus 10. For example, a universally sized loop 30, as is shown in FIG. 1, may fit over substantially all body parts of a user. Alternatively, a small loop 30 may be sized to fit over a user's arm, wrist or ankle but may be too small to fit over a user's thigh or head, in which case a larger loop 30 may be used. The loop 30 may be sufficiently small such that inserting an arm or leg requires the loop 30 to stretch, creating some tension over the body part as the elongated elastic fabric material 12 is wrapped over it. As one having skill in the art can see, a variety of sized loops 30 may be included, all of which are considered within the scope of the present disclosure.

The second end 16 of the elongated elastic fabric material 12 may include a retention device 40 to retain the wrap apparatus 10 in a position about a body part. The retention device 40 may include a variety of devices and systems, such as a hook and loop system and/or a retention clip. Preferably, the retention device 40 is a hook and loop system wherein the hooks of the hook and loop system are located on the retention device 40 and the loops of the hook and loop system are located on the middle portion 18 of the elongated elastic fabric material 12. This may allow the retention device 40 to be removably fixable to the middle portion 18 of the elongated elastic fabric material 12 via the hook and loop system. The loop 30, a hook and loop material of the retention device 40 and a hook and loop material positioned to engage with the of the retention device 40, such as a material located on the middle portion 18, may be formed from one or more elastic materials to stretch and retract with the elongated elastic fabric material 12. Retention devices 40 that include other retention systems may also be removably fixable to the middle portion 18. One benefit of the retention device 40 being removably fixable to the middle portion 18 is that, unlike a belt that is used around a user's waist that only is fixable between the ends, the wrap apparatus 10 is fixable to many points on the elongated elastic fabric material 12. This allows for a snug fit over a user's body part, thereby providing a compressive force on the body part as well as sufficient thermal transfer from a thermal unit that may be housed within the wrap apparatus 10 to the body part.

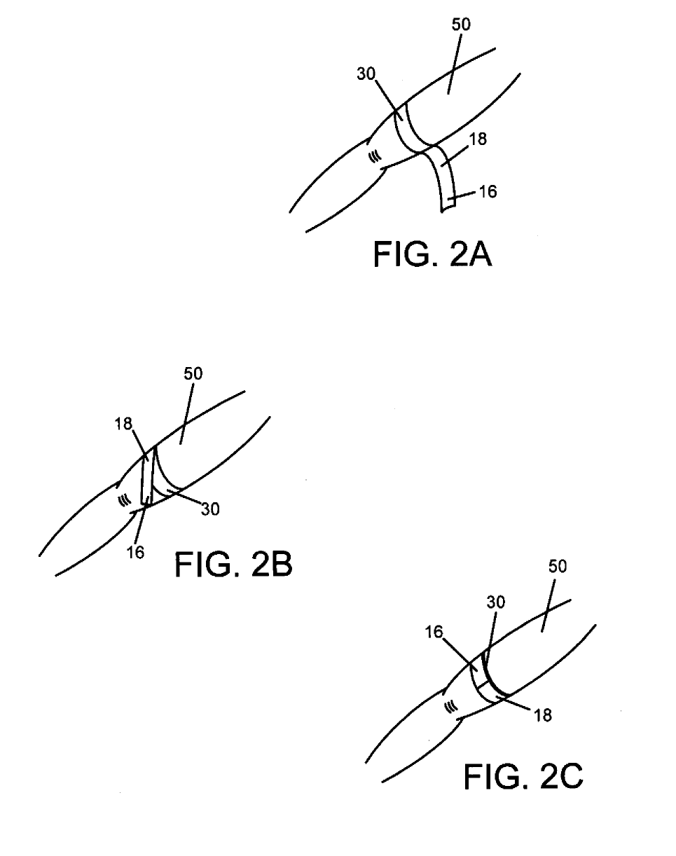

FIGS. 2A-2C are illustrations of a prospective view of a wrap apparatus 10 on a body part 50 in a first exemplary way, in accordance with the first exemplary embodiment of the present disclosure. The wrap apparatus 10 may be wrapped about a body part 50 of a user in at least two ways. The first exemplary way, as illustrated in FIGS. 2A-2C, includes inserting the body part 50 into the loop 30, which positions the loop 30 about the exterior of the body part 50, as shown in FIG. 2A. The elongated elastic fabric material 12 is then wrapped about the body part 50 in overlapping layers until the point where the middle portion 14 of the elongated elastic fabric material 12 is fully wrapped about the body part 50 and the second end 16 of the elongated elastic fabric material 12 is remaining, as shown in FIG. 2B. The second end 16 having the retention device 40 may then be retained by affixing the retention device 40 to the middle portion 14 of the elongated elastic fabric material 12, as is shown in FIG. 2C.

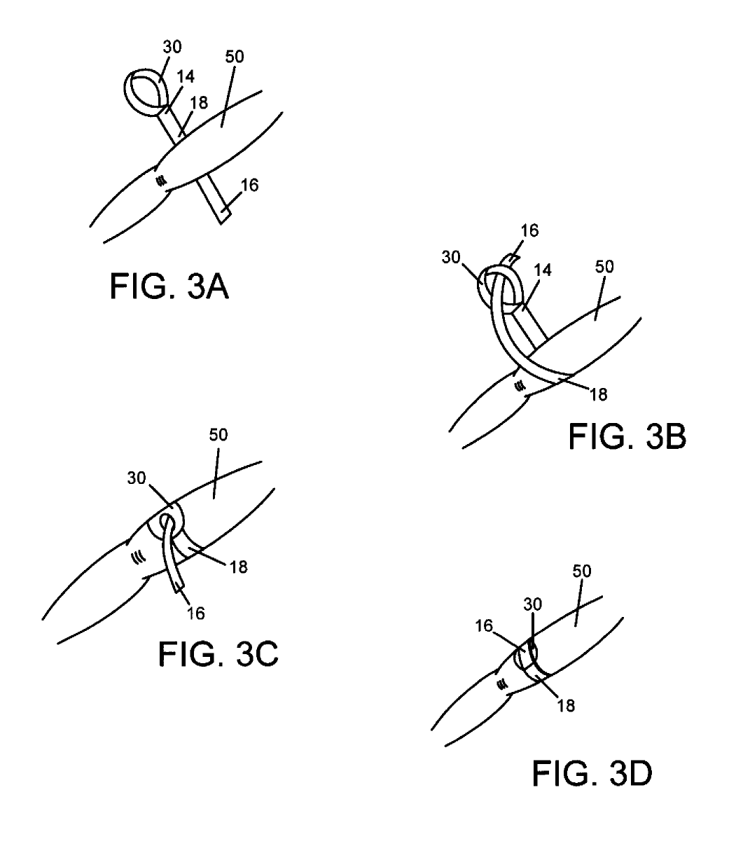

FIGS. 3A-3D are illustrations of a prospective view of a wrap apparatus 10 on a body part 50 in a second exemplary way, in accordance with the first exemplary embodiment of the present disclosure. In the second exemplary way to use the wrap apparatus 10, the middle portion 14 of the elongated elastic fabric material 12 may be placed proximate to the body part 50, as is shown in FIG. 3A. The second end 16 is inserted into the loop 30 whereby the body part 50 is located within the encircling wrap apparatus 10, as is shown in FIG. 3B. The second end 16 may then be pulled through the loop 30, thereby compressing the body part 50 within the wrap apparatus 10, as is shown in FIG. 3C. The remaining middle portion 14 may be wrapped about the body part 50 in overlapping layers until the point where the middle portion 14 is fully wrapped about the body part 50 and the second end 16 is remaining. The second end 16 having the retention device 40 may be retained by affixing the retention device 40 to the middle portion 14 of the elongated elastic fabric material 12, as is shown in FIG. 3D. Although two exemplary ways of using the wrap apparatus 10 are disclosed herein, there may be additional and alternative ways of using the wrap apparatus 10, all of which are considered within the scope of the present disclosure.

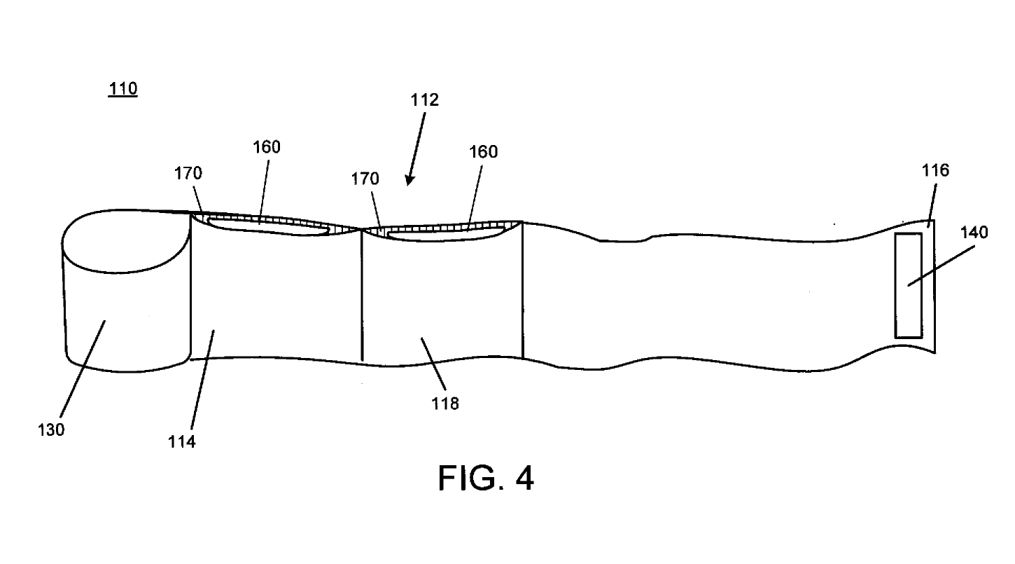

FIG. 4 is an illustration of a prospective view of a wrap apparatus 110 having a thermal pack 160, in accordance with a second exemplary embodiment of the present disclosure. The thermal pack 160 may be any unit that provides a thermal change, such as an ice pack, which is colder than room temperature, or a heating pad that is hotter than room temperature. Any type of hot or cold thermal pack 160 may be used with the wrap apparatus 110. The thermal pack 160 may be encased water, a saline solution, sand, a gel, or any other substance known in the industry for use in a heated and/or cooled pack for application to an injury. The thermal pack 160 may also be reusable, such as an ice pack that is re-freezable or a heat pack that may be re-heated. Accordingly, all components of the wrap apparatus 110, including the thermal packs 160, are designed to be capable of enduring the conditions of a freezer, a refrigerator and a microwave. The wrap apparatus 110 with the thermal pack 160 may provide heat or cold and compression to a body part, as is commonly needed in medical and therapeutic situations, such as with sprained ankles, back injuries and headaches, just to name a few.

The thermal pack 160 may be attached to the elongated elastic fabric material 112. This may include housing the thermal pack 160 within one or more pockets 170 that are formed within the elongated elastic fabric material 112. FIG. 4 illustrates two pockets 170 within the elongated elastic fabric material 112, but any number and any size of pockets 170 may be used to house any number and any size of thermal packs 160. Commonly, the wrap apparatus may include one to twenty small thermal packs 160 or two to three large thermal packs 160, although any number the thermal packs 160 may be used with the wrap apparatus 110. In addition, more than one thermal pack 160 may be used in each of the pockets 170 to provide a lesser or greater thermal change within the wrap apparatus 110.

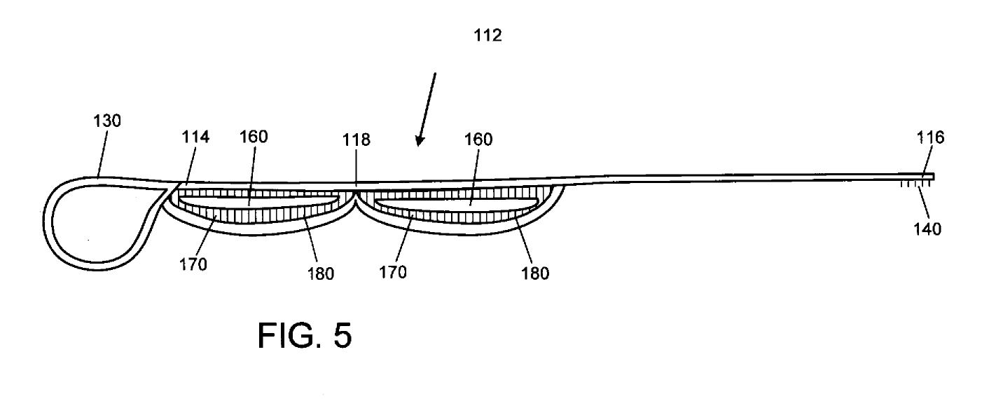

FIG. 5 is an illustration of a top view of a wrap apparatus having a thermal pack, in accordance with the second exemplary embodiment of the present disclosure. The thermal pack 160 may be removably held or permanently held within the pockets 170 of the elongated elastic fabric material 112. For example, the thermal pack 160 may be permanently stitched, sewn within the pockets 170, or the thermal packs 160 may be permanently joined with the elongated elastic band 112, thereby eliminating the need for pockets 170. If a pocket 170 is used with the elongated elastic fabric material 112 to house a thermal pack 160, the pocket 170 may be sealable to removably retain the thermal pack 160 within the pocket 170. For example, a hook and loop system 180 may be used along the opening of the pocket 170 thereby enclosing the at least one thermal pack 160 within the pocket 170, as is shown in FIG. 5. A number of other devices and systems may also be used to seal the pockets 170, such as buttons, snaps and/or a magnetic system, all of which are considered within the scope of this disclosure.

As shown in FIGS. 4 and 5, a plurality of thermal packs 160 may be individually placed within the pockets 170 of the elongated elastic fabric material 112 or a plurality of thermal packs 160 may be placed within one single pocket (not shown). Utilizing a plurality of thermal packs 160 may allow the wrap apparatus 110 to flex and bend more easily along the length and width of the elongated elastic fabric material 112 to more closely matching the contour of the wrapped, distressed body part 50. For example, using a plurality of thermal packs 160 that have been frozen and have a substantial rigidity allows the elongated elastic fabric material 112 to bend around a curved body part, such as a leg or arm. However, when the elongated elastic fabric material 112 with the thermal packs 160 is needed along a substantially linear body part, such as a back, it may be more desirable to use two or three larger thermal packs 160 which may offer less flexibility than smaller thermal packs 160, but may provide for a stronger thermal conduction.

In accordance with the second embodiment, and all additional embodiments of the present disclosure, any component of the wrap apparatus 110 may be constructed from a medical grade, fluid-resistant, stain resistant material. In particular, it may be beneficial to construct the portion of the wrap apparatus 110 that directly abuts the body part from a medical grade, fluid-resistant, stain resistant material, thereby preventing perspiration, blood, or another bodily fluid from soaking through the wrap apparatus 110. In addition, a medical grade, fluid-resistant, stain resistant material may also prevent condensation from a thermal pack 160 from leaking through the wrap apparatus 110. For example, constructing the pockets 170 from a medical grade, fluid-resistant, stain resistant material may allow for the thermal packs 160 to abut a body part with only the medical grade, fluid-resistant, stain resistant material acting as a barrier between the thermal packs 160 and the body part. Convenience, efficiency and ease of use of the wrap apparatus 110 may be enhanced by constructing all components out of microwaveable-safe and washer machine-safe materials.

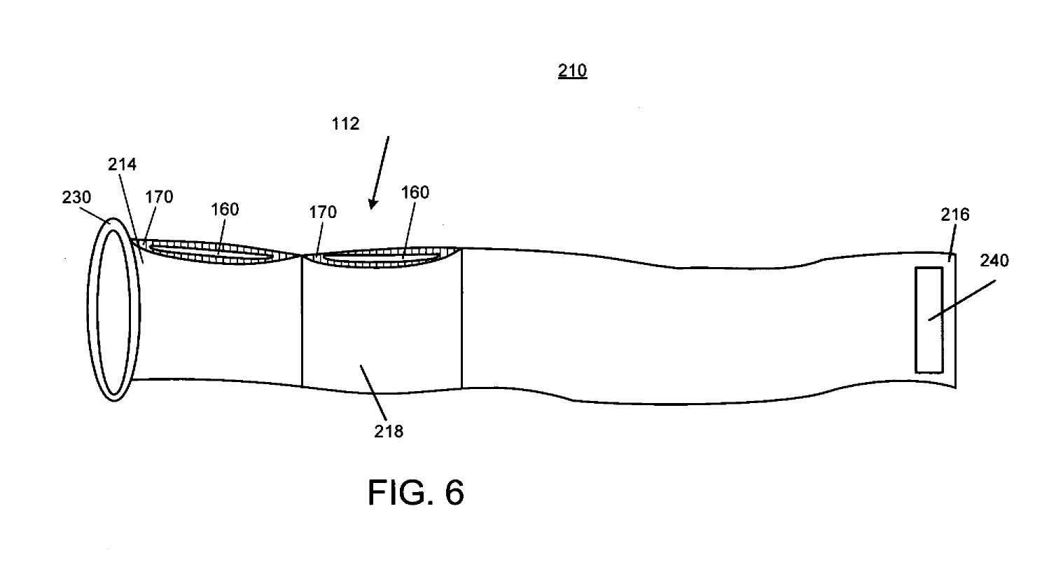

FIG. 6 is an illustration of a prospective view of a wrap apparatus 210, in accordance with a third exemplary embodiment of the present disclosure. The wrap apparatus 210 of the third exemplary embodiment is substantially similar to the wrap apparatus 10, 110 or the first and second exemplary embodiments, and may include any of the features discussed with regards to the first or second embodiments. The wrap apparatus 210 of the third exemplary embodiment includes a loop 230 that is positioned, with respect to the elongated elastic fabric material 220, whereby a central axis of the loop 230 is perpendicular to a planar face of the elongated elastic fabric material 220. This configuration may allow the wrap apparatus 210 to be used on a body part without the twisting or bunching of materials when the wrap apparatus 210 is used in accordance to the second exemplary way as illustrated in FIGS. 3A-3D.

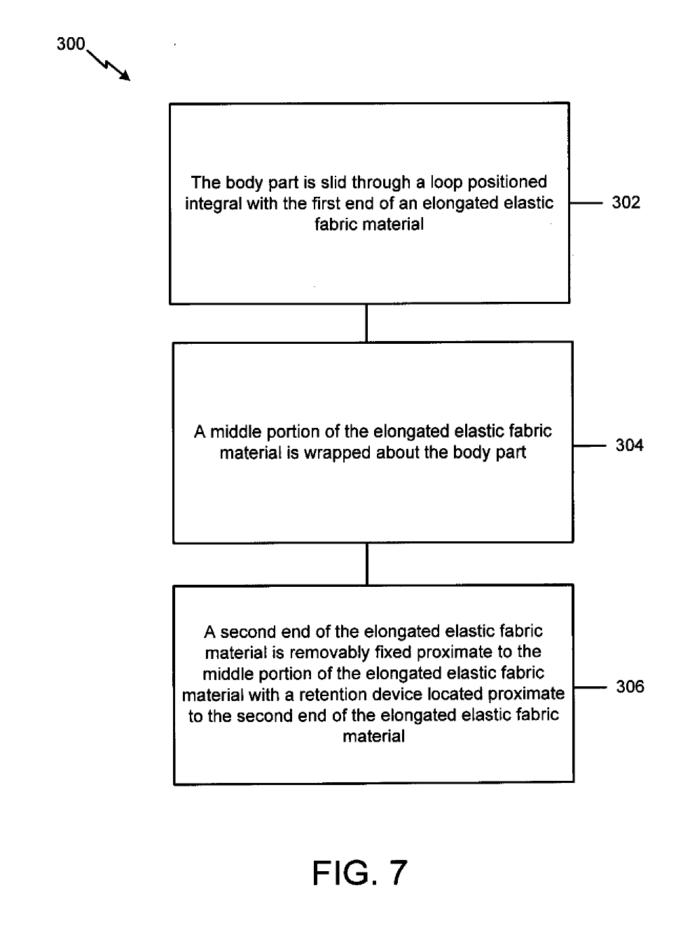

FIG. 7 is an illustration of a flowchart 300 illustrating a method of wrapping a body part with a wrap apparatus of FIG. 1, in accordance with the first exemplary embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 302, the body part is slid through a loop positioned integral with the first end of an elongated elastic fabric material. A middle portion of the elongated elastic fabric material is wrapped about the body part (Block 304). A second end of the elongated elastic fabric material is removably fixed proximate to the middle portion of the elongated elastic fabric material with a retention device located proximate to the second end of the elongated elastic fabric material (Block 306)

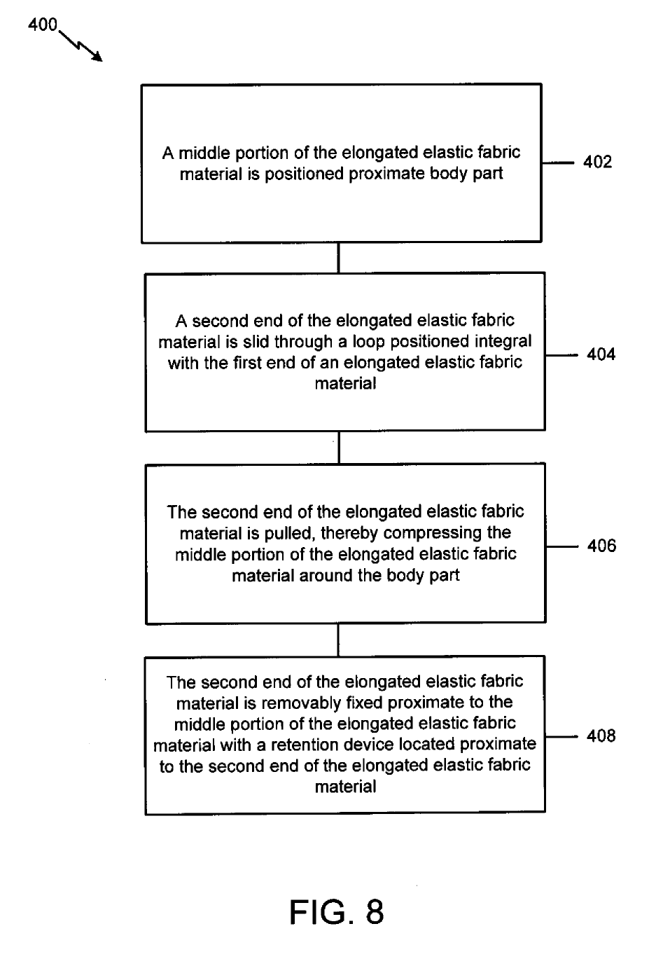

FIG. 8 is an illustration of a flowchart 400 illustrating a method of wrapping a body part with a wrap apparatus of FIG. 1, in accordance with the first exemplary embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 402, a middle portion of an elongated elastic fabric material is positioned proximate a body part. A second end of the elongated elastic fabric material is slid through a loop positioned integral with the first end of an elongated elastic fabric material (Block 404). The second end of the elongated elastic fabric material is pulled, thereby compressing the middle portion of the elongated elastic fabric material around the body part (Block 406). The second end of the elongated elastic fabric material is removably fixed proximate to the middle portion of the elongated elastic fabric material with a retention device located proximate to the second end of the elongated elastic fabric material (Block 408).

In either of the methods discussed with respect to FIGS. 7 and 8, the flowcharts 300, 400 may include any number of additional steps. For example the step of wrapping the middle portion of the elongated elastic fabric material about the body part may further include stretching the elongated elastic fabric material. Furthermore, at least one thermal pack may be placed within at least one pocket formed within the middle portion of the elongated elastic fabric material. The thermal properties of the at least one thermal pack may be changed by freezing the at least one thermal pack and/or heating the at least one thermal pack.

It should be emphasized that the above-described embodiments of the present disclosure, particularly, any “preferred” embodiments, are merely possible examples of implementations, merely set forth for a clear understanding of the principles of the disclosed system and method. Many variations and modifications may be made to the above-described embodiments of the disclosure without departing substantially from the spirit and principles of the disclosure. All such modifications and variations are intended to be included herein within the scope of this disclosure and protected by the following claims.

What is claimed is:

1. A wrap apparatus comprising:
an elongated elastic fabric material having a first end, a second end and a middle portion located between the first end and second end;
a loop integral with the first end of the elongated elastic fabric material; and
a retention device located proximate to the second end of the elongated elastic fabric material, wherein the retention device is removably fixable to the middle portion of the elongated elastic fabric material.

2. The wrap apparatus of claim 1, wherein the loop is formed with the elongated elastic fabric material.

3. The wrap apparatus of claim 1, wherein the loop is formed from a second material and is affixed integrally to the first end of the elongated elastic fabric material.

4. The wrap apparatus of claim 1, wherein the retention device further comprises a hook and loop system.

5. The wrap apparatus of claim 1, further comprising at least one thermal pack located with at least one pocket formed within the middle portion of the elongated elastic fabric material.

6. The wrap apparatus of claim 5, wherein the at least one thermal pack provides at least one of a temperature hotter than room temperature and colder than room temperature.

7. The wrap apparatus of claim 5, wherein the at least one pocket is sealable with a hook and loop system, thereby enclosing the at least one thermal pack within the at least one pocket.

8. The wrap apparatus of claim 5, wherein at least a portion of the at least one pocket formed within the middle portion of the elongated elastic fabric material is constructed from a medical grade, fluid-resistant, stain resistant material.

9. The wrap apparatus of claim 1, wherein the elongated elastic fabric material, the loop and the retention device are at least one of microwaveable and machine washable.

10. The wrap apparatus of claim 1, wherein the loop is positioned, with respect to the elongated elastic fabric material, whereby a central axis of the loop is perpendicular to a planar face of the elongated elastic fabric material.

11. The wrap apparatus of claim 1, wherein the retention device further comprises a hook and loop system, wherein a plurality of hooks of the hook and loop system are located on the retention device and a plurality of loops of the hook and loop system are located on the middle portion of the elongated elastic fabric material.

12. The wrap apparatus of claim 1, wherein at least one of the loop, a hook and loop material of the retention device and a hook and loop material positioned to engage with the of the retention device are formed from at least one elastic material.

13. A method of wrapping a body part with a wrap apparatus, the method comprising the steps of:
sliding the body part through a loop positioned integral with the first end of an elongated elastic fabric material;
wrapping a middle portion of the elongated elastic fabric material about the body part; and
removably fixing a second end of the elongated elastic fabric material proximate to the middle portion of the elongated elastic fabric material with a retention device located proximate to the second end of the elongated elastic fabric material.

14. The method of wrapping a body part with a wrap apparatus of claim 13, wherein the step of wrapping the middle portion of the elongated elastic fabric material about the body part further comprising stretching the elongated elastic fabric material.

15. The method of wrapping a body part with a wrap apparatus of claim 13, further comprising the step of placing at least one thermal pack within at least one pocket formed within the middle portion of the elongated elastic fabric material.

16. The method of wrapping a body part with a wrap apparatus of claim 15, further comprising the step of changing the thermal properties of the at least one thermal pack by at least one of freezing the at least one thermal pack and heating the at least one thermal pack.

17. A method of wrapping a body part with a wrap apparatus, the method comprising the steps of:
positioning a middle portion of an elongated elastic fabric material proximate a body part;
sliding a second end of the elongated elastic fabric material through a loop positioned integral with the first end of an elongated elastic fabric material;
pulling the second end of the elongated elastic fabric material, thereby compressing the middle portion of the elongated elastic fabric material around the body part; and
removably fixing the second end of the elongated elastic fabric material proximate to the middle portion of the elongated elastic fabric material with a retention device located proximate to the second end of the elongated elastic fabric material.

18. The method of wrapping a body part with a wrap apparatus of claim 17, wherein the step of wrapping the middle portion of the elongated elastic fabric material about the body part further comprising stretching the elongated elastic fabric material.

19. The method of wrapping a body part with a wrap apparatus of claim 17, further comprising the step of placing at least one thermal pack within at least one pocket formed within the middle portion of the elongated elastic fabric material.

20. The method of wrapping a body part with a wrap apparatus of claim 19, further comprising the step of changing the thermal properties of the at least one thermal pack by at least one of freezing the at least one thermal pack and heating the at least one thermal pack.
